(19) United States
(12) Patent Application Publication
(10) Pub. No.: US 2017/0137426 A1
(43) Pub. Date: May 18, 2017

(54) HETEROCYCLIC COMPOUNDS AS AXL INHIBITORS

(71) Applicant: CHANGZHOU JIEKAI PHARMATECH CO., LTD., Changzhou, Jiangsu (CN)

(72) Inventors: Jintao Zhang, Naperville, IL (US); Yibin Xiang, Dracut, MA (US); Wen Xu, Shanghai (CN); Shnzhon Jian, Shanghai (CN)

(73) Assignee: Changzhou Jiekai Pharmatech Co., Ltd., Changzhou, Jiangsu (CN)

(21) Appl. No.: 15/127,522

(22) PCT Filed: Mar. 28, 2014
(86) PCT No.: PCT/CN2014/074248

§ 371 Date: Sep. 20, 2016

(51) Int. Cl. C07D 487/04; A61K 31/519; A61K 31/55; A61K 45/06

(52) U.S. Cl. CPC C07D 487/04; A61K 45/06; A61K 31/519; A61K 31/55

(57) ABSTRACT

Compounds of Formula I and their uses of effective AXL inhibitors and for the treatment of physical condition mediated by AXL.

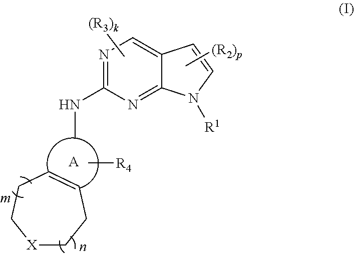

### FIELD OF THE INVENTION

The present application is in general in the field of medicinal chemistry and specifically related to compounds that are protein kinase inhibitors, such as AXL inhibitors. These compounds are useful for treating disease and conditions (e.g., cancers) that are mediated by such protein kinases as AXL.

### BACKGROUND OF THE INVENTION

Human genome contains 20 families of 58 receptor tyrosine kinases (RTKs) that undergo dimerization, autophosphorylation, and activation upon ligand binding, consequently activating downstream intracellular signaling cascades. RTKs regulate diverse cellular processes such as mitogenesis, cell cycle, growth, differentiation and development, survival and apoptosis, cell shape and adhesion, migration, and angiogenesis. Dysregulation or mutations in RTKs can induce aberrant activity and lead to a broad spectrum of human diseases (Hubbard, S. and Till, J., Annu Rev Biochem, 69: 373-398, 2000). TAM is a subfamily of RTKs comprised of the related TYRO-3, AXL, and MER. The TAM RTKs are defined by unique tandem immunoglobin-like repeats and dual fibronectin type III repeats in the extracellular region, and can be activated by a common ligand of growth arrest-specific 6 (Gas 6).

AXL is ubiquitously expressed in a wide variety of organs and cells, including the hippocampus and cerebellum, monocytes, macrophages, platelets, endothelial cells, heart, skeletal muscle, liver, kidney, and testis. Activation of AXL in cells leads to activating the anti-apoptotic/survival PI3K/Akt and the mitogenic Ras/Raf/Mek/Erk cascade signaling pathways that promote cell growth, proliferation, and motility in general (Verma, A., et al, Mol Cancer Ther, 10:1763-1773, 2011). In cells and tissues, these AXL-stimulated intracellular signaling pathways regulate different aspects of physiological functions. Angiogenesis is the formation of new blood vessels by endothelial cells. Gas 6 is widely expressed in human endothelial and vascular smooth muscle cells. Activation of AXL by Gas 6 in these cells regulates angiopoietin signaling system and stimulates the proliferation and migration of endothelial and vascular smooth muscle cells, thereby controlling the tube formation and vascular regression, vascular homeostasis, and angiogenesis (Fridell, Y, et al, J Biol Chem, 273:7123-6, 1998; Holland, S., et al, Cancer Res, 65:9294-303, 2005). AXL signaling also plays important roles in immunity (Lu, Q. and Lemke, G., Science, 293:306-311, 2001; Scott, R., et al, Nature, 411:207-211, 2001), platelet function (Angelillo-Scherrer, A., et al, Nat Med, 7:215-221, 2001), and kidney functions (Yanagita, M., et al, J Clin Invest, 110:239-246, 2002).

Aberrant activation of AXL is associated with many aspects of tumorigenesis. The intracellular signaling pathways activated by AXL are commonly found to be hyper-activated and hijacked by tumors to drive cancer cell survival and proliferation. In addition, by regulating angiogenesis in tumor environment, AXL promotes tumor growth, invasiveness, and metastasis. AXL was originally identified as a protein encoded by a transforming gene that over-expresses in primary human myeloid leukemia cells (O'Bryan, J., et al., Mol Cell Bio, 11:5016-5031, 1991). Subsequently, activation of AXL by over-expression is frequently discovered in numerous types of human cancers and found to play an essential role in cancer development and maintenance. AXL over-expression was observed in 55% ductal adenocarcinoma of the pancreas. These patients are significantly associated with lymph node metastasis and have a shorter median survival of 12 months compared with AXL-negative cancers of 18 months (Koorstra, J., et al, Cancer Biol Ther, 8:618-26, 2009). In glioblastomas, over-expression of AXL changes cellular morphology and increases filopodia by regulating cytoskeleton actin to favor cancer cell motility and invasion (Vajkoczy, P., et al., PNAS, 15:5799-804, 2006). In breast cancer models, ectopic expression of AXL significantly transforms weakly metastatic MCF7 cells into highly invasive ones (Zhang, Y., et al, Cancer Res, 68:1905-15, 2008). Furthermore, in clinical patient samples of non-small cell lung cancer, AXL protein over-expression has been statistically associated with lymph node involvement and advanced clinical stage of disease (Shieh, Y., et al, Neoplasia 7:1058-64, 2005).

There remain tremendously unmet clinical needs in treating human cancers as a result of the limited efficacy, prohibitive toxicities, or in many cases both, of current portfolio of medicines. Due to drug resistance, majority cancer patients are treated with regimens consisting of several lines of drugs with distinct pharmacological mechanism of action. An emerging concept of the targeted anticancer therapy aims to develop specific small molecule chemicals or biologic proteins to inhibit aberrantly-activated kinases in cancer cells. This approach has resulted in enormous success by developing therapies targeting RTKs such as EGFR, HER2, PDGF, IGF, MET, etc., that are being adopted in clinical treatment for various cancers. Like these validated RTK drug targets, AXL has demonstrated a similarly strong link to human cancers. In several preclinical cancer models, inhibition of AXL activity by pharmacological, cellular, or genetic approaches including small molecule compounds, dominant negative protein, or siRNA inhibits cancer cell proliferation, induces cell apoptosis, suppresses tumor angiogenesis, and reduces tumor invasive capacity. These results establish AXL as an attractive and valuable target for the discovery and development of new therapeutic agents in human cancers including lung cancer, myeloid leukemia, astrocytoma, uterine cancer, ovarian cancer, colorectal carcinoma, esophageal adenocarcinoma, glioblastoma, melanoma, prostate cancer, breast cancer, osteosarcoma, renal cell carcinoma, thyroid cancer, gastrointestinal stromal tumors, gastric cancer, hepatocellular carcinoma, kaposi sarcoma, pancreatic ductal adenocarcinoma, prostate cancer, and endometrial cancer.

In addition, due to AXL's role in regulating angiogenesis, immune cell and platelet functions, etc., AXL inhibition also would be of benefit in the treatment of various complications and diseases such as asthma, chronic bronchitis, chronic obstructive pulmonary disease, adult respiratory distress syndrome, infant respiratory distress syndrome, cough, chronic obstructive pulmonary, adult respiratory distress syndrome, ulcerative colitis, Crohn's disease, hypersecretion of gastric acid, bacterial-, fungal-, or viral-induced sepsis or septic shock, endotoxic shock, spinal cord trauma, head injury, neurogenic inflammation, pain, reperfusion injury of the brain, psoriatic arthritis, rheumatoid arthritis, alkylosing spondylitis, osteoarthritis, inflammation, cytokine-mediated chronic tissue degeneration, thrombosis and the complications associated with thrombosis, macular degeneration, cataracts, diabetic retinopathy, glomerulonephritis, diabetic nephropathy, and renal plant rejection.

### BRIEF DESCRIPTION OF THE INVENTION

The present invention in general provides compounds that exhibit surprisingly superior effect on inhibiting AXL, pharmaceutical compositions containing the compounds and uses thereof.

In one aspect, the present invention provides compounds or pharmaceutically acceptable salts thereof that, among others, are surprisingly effective AXL inhibitors. These compounds are of Formula I as shown below:

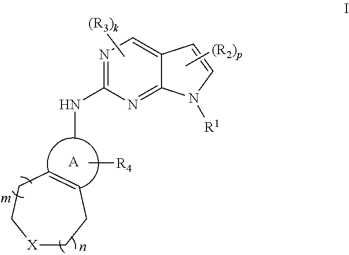

### In Formula I,

A is a 5- or 6-membered aryl or heteroaryl, and is optionally substituted with one or more R₄ groups;
p is 0, 1, or 2; k is 0 or 1;
each of m and n independently is 0, 1, 2, or 3, and the sum of m and n is less than 4;
X is CHR₅ or NR₆;
R₁ is hydrogen, aryl, heteroaryl, cycloalkyl, or heterocyclyl, and is optionally substituted with 1 to 4 Rₐ groups;
each of R₂ and R₃ independently is halogen, alkyl, alkenyl, alkynyl, haloalkyl, hydroxyl, hydroxylalkyl, alkoxy, alkenyloxy, alkynyloxy, carbonyl, carboxyl, cyano, amino, nitrile, sulfonyl, sulfinyl, sulfhydryl, aryl, cycloalkyl, heteroaryl, or heterocyclyl;
each optional R₄ group independently is halogen, alkyl, alkenyl, alkynyl, haloalkyl, hydroxyl, hydroxylalkyl, alkoxy, alkenyloxy, alkynyloxy, carbonyl, carboxyl, cyano, amino, nitrile, sulfonyl, sulfinyl, sulfhydryl, aryl, cycloalkyl, heteroaryl, or heterocyclyl;
R₅ is hydrogen, amine, alkylamine, cyclic amine, heterocyclyl, alkyl, alkenyl, alkynyl, cycloalkyl, aryl, heteroaryl, nitrile, sulfonyl, sulfinyl, sulfhydryl, halogen, haloalkyl, hydroxyl, hydroxyalkyl, alkoxy, alkenyloxy, alkynyloxy, carbonyl, or carboxyl;
R₆ is hydrogen, alkyl, alkenyl, alkynyl, cycloalkyl, aryl, CN, heteroaryl, or heterocyclyl; or
each optional Rₐ group independently is halogen, alkyl, alkenyl, alkynyl, cycloalkyl, aryl, heteroaryl, heterocyclyl, alkoxy, alkenyloxy, alkynyloxy, cycloalkyloxy, aryloxy, heteroaryloxy, heterocyclyloxy, alkylamino, amino carbonyl, acyl, carbonyl, carboxyl, amino, cyano, cyanato, nitrile, sulfonyl, sulfinyl, or sulfhydryl.

In some embodiments, A is a 6- or 5-membered heteroaryl having 1 to 3 heteroatoms each of which independently is O, S, or N, and A is optionally substituted with 1 to 3 R₄ groups.

In some other embodiments, A is

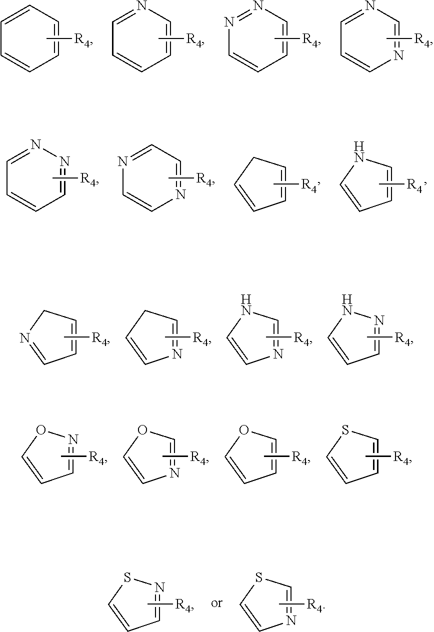

For example, A is

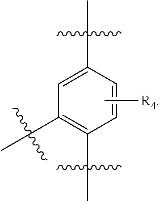

In some embodiments, R₁ is aryl or heteroaryl optionally substituted with 1 to 4 Rₐ groups. In a narrower set of embodiments, R₁ is

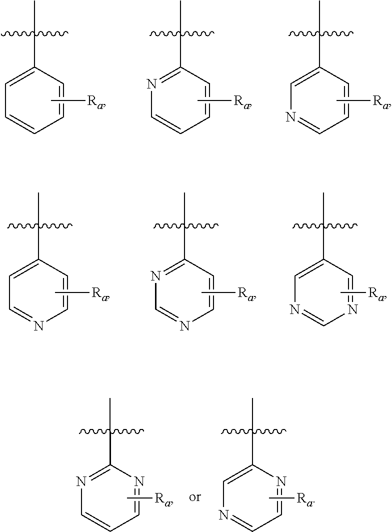

In some embodiments, each Rₐ independently is halogen, optionally substituted lower alkyl, optionally substituted aryl, optionally substituted heteroaryl, optionally substituted cycloalkyl, optionally substituted amino, cyano, cyanato, optionally substituted alkoxy, optionally substituted alkenyloxy, optionally substituted alkynyloxy, optionally substituted cycloalkyloxy, optionally substituted aryloxy, amino carbonyl, or hydroxyl. Examples of such Rₐ include F, Cl, Br, methyl, ethyl, propyl, isopropyl, butyl, isobutyl, t-butyl, optionally substituted phenyl, optionally substituted morphalinyl, optionally substituted piperazinyl, optionally substituted pyridine, methoxyl, ethoxy, propoxy, isopropoxy, optionally substituted phenoxyl, optionally substituted cyclohexyloxy, and optionally substituted cyclopentyloxy.

In some embodiments, R₅ is

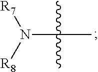

each of R₇ and R₈ independently is hydrogen, optionally substituted lower alkyl, optionally substituted cycloalkyl, optionally substituted aryl, optionally substituted heteroaryl, cyano, optionally substituted alkoxy, optionally substituted alkenyloxy, hydroxyl, carbonyl, carboxyl, or hydroxylalkyl; or R₇ and R₈, together with the nitrogen atom to which they are attached, form a 4- to 8-membered optionally substituted heterocyclyl or heteroaryl. Specific examples of R₅ include

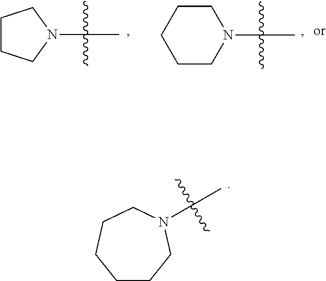 [SMILES: *N1CCCC1 ; *N1CCCCC1 ; *N1CCCCCC1]

In some embodiments, R₆ is optionally substituted alkyl or cycloalkyl. Specific examples of R₆ include methyl, ethyl, propyl, isopropyl, butyl, isobutyl, t-butyl, cyclopropyl, cyclopentyl, and cyclohexyl.

In some embodiments, m is 1 and n is 1; m is 0 and n is 1; m is 0 and n is 2; m is 0 and n is 3; or m is 1 and n is 2.

In some other embodiments, the compounds of this invention are of Formula II as shown below:

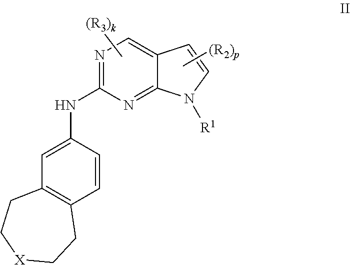

In Formula II, X, R₁, R₂, R₃, p, and k are as described above.

In a narrower set of embodiments,

k is 0 and p is 0;

R₁ is

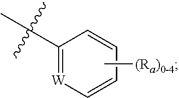

W is CR_{b}, CH, or N; each of Rₐ and R_{b} independently is halogen, optionally substituted lower alkyl, optionally substituted aryl, optionally substituted heteroaryl, optionally substituted cycloalkyl, optionally substituted optionally substituted alkoxy, optionally substituted cycloalkyloxy, optionally substituted aryloxy, amino, amino carbonyl, cyano, cyanato, or hydroxyl; or

X is CHR₅ or NR₆; R₅ is

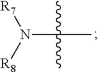

each of R₇ and R₈ independently is hydrogen or alkyl; or R₇ and R₈, together with the nitrogen atom to which they are attached, form a 4- to 8-membered heterocyclyl or heteroaryl; and R₆ is optionally substituted lower alkyl or cycloalkyl.

Still in a narrower set of embodiments, R_{b} is halogen or optionally substituted lower alkyl; and each Rₐ independently is halogen, optionally substituted aryl, optionally substituted heteroaryl, optionally substituted alkoxy, optionally substituted cycloalkyloxy, or optionally substituted aryloxy. Specific examples of Rₐ in Formula II include, but are not limited to, F, Cl, Br, methyl, ethyl, propyl, isopropyl, butyl, isobutyl, t-butyl, phenyl, methoxyl, ethoxy, propoxy, isopropoxy, phenoxyl, cyclohexyloxy, and cyclopentyloxy.

Specific examples of the compounds of this invention include
7-(2-isopropoxyphenyl)-N-(7-(pyrrolidin-1-yl)-6,7,8,9-tetrahydro-5H-benzo[7]annulen-2-yl)-7H-pyrrolo[2,3-d]pyrimidin-2-amine;
7-(3-isopropoxyphenyl)-N-(7-(pyrrolidin-1-yl)-6,7,8,9-tetrahydro-5H-benzo[7]annulen-2-yl)-7H-pyrrolo[2,3-d]pyrimidin-2-amine;
7-(4-isopropoxyphenyl)-N-(7-(pyrrolidin-1-yl)-6,7,8,9-tetrahydro-5H-benzo[7]annulen-2-yl)-7H-pyrrolo[2,3-d]pyrimidin-2-amine;
7-(2-phenoxyphenyl)-N-(7-(pyrrolidin-1-yl)-6,7,8,9-tetrahydro-5H-benzo[7]annulen-2-yl)-7H-pyrrolo[2,3-d]pyrimidin-2-amine;
7-(2-(cyclohexyloxy)phenyl)-N-(7-(pyrrolidin-1-yl)-6,7,8,9-tetrahydro-5H-benzo[7]annulen-2-yl)-7H-pyrrolo[2,3-d]pyrimidin-2-amine;
N-(7-(pyrrolidin-1-yl)-6,7,8,9-tetrahydro-5H-benzo[7]annulen-2-yl)-7-(2-(o-tolyloxy)phenyl)-7H-pyrrolo[2,3-d]pyrimidin-2-amine;
N-isopropyl-2-(2-((7-(pyrrolidin-1-yl)-6,7,8,9-tetrahydro-5H-benzo[7]annulen-2-yl)amino)-7H-pyrrolo[2,3-d]pyrimidin-7-yl)benzamide;
7-(4-chloro-2-isopropoxyphenyl)-N-(7-(pyrrolidin-1-yl)-6,7,8,9-tetrahydro-5H-benzo[7]annulen-2-yl)-7H-pyrrolo[2,3-d]pyrimidin-2-amine;
7-(2-isopropoxy-4-methoxyphenyl)-N-(7-(pyrrolidin-1-yl)-6,7,8,9-tetrahydro-5H-benzo[7]annulen-2-yl)-7H-pyrrolo[2,3-d]pyrimidin-2-amine;
7-(3-isopropoxy-[1,1′-biphenyl]-4-yl)-N-(7-(pyrrolidin-1-yl)-6,7,8,9-tetrahydro-5H-benzo[7]annulen-2-yl)-7H-pyrrolo[2,3-d]pyrimidin-2-amine;
7-([1,1′-biphenyl]-4-yl)-N-(7-(pyrrolidin-1-yl)-6,7,8,9-tetrahydro-5H-benzo[7]annulen-2-yl)-7H-pyrrolo[2,3-d]pyrimidin-2-amine;
7-(2′-methyl-[1,1′-biphenyl]-4-yl)-N-(7-(pyrrolidin-1-yl)-6,7,8,9-tetrahydro-5H-benzo[7]annulen-2-yl)-7H-pyrrolo[2,3-d]pyrimidin-2-amine;
7-(3-isopropoxypyridin-2-yl)-N-(7-(pyrrolidin-1-yl)-6,7,8,9-tetrahydro-5H-benzo[7]annulen-2-yl)-7H-pyrrolo[2,3-d]pyrimidin-2-amine; and
N-(7-(2-isopropoxyphenyl)-7H-pyrrolo[2,3-d]pyrimidin-2-yl)-3-methyl-2,3,4,5-tetrahydro-1H-benzo[d]azepin-7-amine.

The compounds of this invention also include 7-(2-isopropoxyphenyl)-N-(1-(1-methylpiperidin-4-yl)-1H-pyrazol-4-yl)-7H-pyrrolo[2,3-d]pyrimidin-2-amine.

In another aspect, the present invention provides pharmaceutical compositions each comprising a compound of this invention as described above (e.g., a compound of Formula I disclosed herein) and a pharmaceutically acceptable carrier. In some embodiments, each of the compositions further includes an additional therapeutic agent. Examples of such therapeutic agent include, but are not limited to, a chemotherapeutic or anti-proliferative agent, an anti-inflammatory agent, an immunomodulatory or immunosuppressive agent, an agent for treating a neurological disorder, an agent for treating cardiovascular disease, an agent for treating destructive bone disorders, an agent for treating liver disease, an anti-viral agent, an agent for treating blood disorders, an agent for treating diabetes, and an agent for treating immunodeficiency disorders.

Still in another aspect, the present invention relates to a method of treating a disease, disorder, or condition mediated by AXL or associated with AXL activity in a patient, which comprises administering to the patient in need thereof a therapeutically effective amount of a compound or pharmaceutical composition of this invention. Yet still another aspect of this invention provides using a compound of this invention for the manufacture of a medicament for the treatment of the disease, disorder, or condition mediated by AXL or associated with AXL activity.

Such a disease, disorder, or condition is usually alleviated by a decrease of AXL activity. Examples of such a disease, disorder, or condition include but are not limited to cancer, asthma, chronic bronchitis, chronic obstructive pulmonary disease, adult respiratory distress syndrome, infant respiratory distress syndrome, cough, chronic obstructive pulmonary, adult respiratory distress syndrome, ulcerative colitis, Crohn's disease, hypersecretion of gastric acid, bacterial-, fungal-, or viral-induced sepsis or septic shock, endotoxic shock, spinal cord trauma, head injury, neurogenic inflammation, pain, reperfusion injury of the brain, psoriatic arthritis, rheumatoid arthritis, alkylosing spondylitis, osteoarthritis, inflammation, cytokine-mediated chronic tissue degeneration, thrombosis and the complications associated with thrombosis, macular degeneration, cataracts, diabetic retinopathy, glomerulonephritis, diabetic nephropathy, and renal plant rejection.

In some embodiments, such a disease, disorder, or condition is a cancer. For example, such a cancer is lung cancer, myeloid leukemia, astrocytoma, uterine cancer, ovarian cancer, colorectal carcinoma, esophageal adenocarcinoma, glioblastoma, melanoma, prostate cancer, breast cancer, osteosarcoma, renal cell carcinoma, thyroid cancer, gastrointestinal stromal tumors, gastric cancer, hepatocellular carcinoma, kaposi sarcoma, pancreatic ductal adenocarcinoma, prostate cancer, or endometrial cancer.

The invention also provides kits comprising a compound disclosed herein or a pharmaceutically acceptable salt, solvate, or prodrug thereof, packaging, and instructions for use thereof. Such kits can be used for the treatment or prevention in an individual of a disease or condition mediated by AXL. In some embodiments, the kit comprises a pharmaceutical formulation which includes a compound of this invention (e.g., a compound of Formula I) and packaging.

Set forth below is a detailed description of the compounds of this invention, methods and processes for making, testing, and using these compounds which also constitute part of this invention.

### DETAILED DESCRIPTION OF THE INVENTION

### Definitions

As use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X.”

As used herein, the word “or” has the meaning of both “or” and “and” and is equivalent to “and/or”—unless otherwise specifically limited to just “or.”

As used herein, the term “halo” or “halogen,” by itself or as part of another substituent (e.g, haloalkyl), refers to and includes fluoro, chloro, bromo, or iodo.

As used herein, the term “alkyl,” by itself or as part of another substituent (which usually takes the short form of “alk,” e.g., alkoxy), refers to and includes saturated linear (i.e. unbranched) or branched hydrocarbon radicals, having the number of carbon atoms designated (e.g., C₁₋₁₀ means one to ten carbons). Particular alkyl groups include those having 1 to 10 carbon atoms (a “C₁₋₁₀ alkyl”). More particular alkyl groups are those having 1 to 6 carbon atoms (a “C₁₋₆ alkyl”), 1 to 4 carbon atoms (a “C₁₋₄ alkyl”), 1 to 3 carbon atoms (a “C₁₋₃ alkyl”) or 1 to 2 carbon atoms (a “C₁₋₂ alkyl”). Examples of “C₁₋₁₀ alkyl” include, but are not limited to, methyl, ethyl, n-propyl, isopropyl, n-butyl, isobutyl, sec-butyl, tert-butyl, n-pentyl, n-hexyl, n-heptyl, n-octyl, n-nonyl, n-decyl and the like. An alkyl group can be optionally substituted with such substituents as halogen, cyano, amino, hydroxyl etc. As used herein, the term “lower alkyl” refers to alkyl of 1 to 6 carbon atoms which are optionally substituted with one or more appropriate substituents such as halogen, amino, cyano, or hydroxyl.

As used herein, the term “alkenyl,” by itself or as part of another substituent, refers to and includes unsaturated linear (i.e. unbranched) or branched hydrocarbon radicals containing at least one carbon-carbon double bond, having the number of carbon atoms designated (e.g., C₂₋₁₀ means two to ten carbons). Particular alkenyl groups are those having 2 to 10 carbon atoms (i.e., a “C₂₋₁₀ alkenyl”). More particular alkenyl groups are those having 2 to 8 carbon atoms (a “C₂₋₈ alkenyl”) or 2 to 6 carbon atoms (a “C₂₋₆ alkenyl”). Examples of “C₂₋₁₀ alkenyl” include, but are not limited to, ethenyl, 1-propenyl, 2-propenyl, 1-methylethenyl, 2-methyl-1-propenyl, 2-methyl-2-propenyl, 2-methyl-3-butenyl, 3-methyl-3-butenyl, 1,2-dimethyl-1-propenyl, and 1,2-dimethyl-2-propenyl. As used herein, the term “lower alkenyl” refers to alkenyl of 1 to 6 carbon atoms which are optionally substituted with one or more appropriate substituents such as halogen, amino, cyano, or hydroxyl.

As used herein, the term “aminocarbonyl” refers to NRR′—C(═O)— wherein each of R and R′ independently can be hydrogen, lower (e.g., C₁₋₆) alkyl or alkenyl which may be optionally substituted with halogen or cyano.

As used herein, the term “heteroatom” refers to “S,” “O” or “N” in a ring which can be saturated, unsaturated, or aromatic. The “N” heteroatom can be optionally subtitled with alkyl or alkenyl.

As used herein, the term “cycloalkyl” or “cyclyl”, by itself or as part of another substituent (e.g., cycloalkyloxy), refers to and includes saturated monocyclic hydrocarbon radicals, having the number of carbon atoms designated (e.g., C₃₋₁₀ means three to ten carbons). Particular examples of cycloalkyl or cyclyl groups include those having 3 to 10 carbon atoms (a “C₃₋₁₀ cycloalkyl”). More particular cycloalkyl groups include those having 3 to 8 carbon atoms (a “C₃₋₈ cycloalkyl”), 3 to 6 carbon atoms (a “C₃₋₆ cycloalkyl”) or 4 to 5 carbon atoms (a “C₄₋₅ cycloalkyl”). Examples of “C₃₋₁₀ cycloalkyl” include, but are not limited to, cyclopropyl, cyclopentyl, cyclohexyl, and the like.

As used herein, the term “alkoxy” refers to an alkyl group linked by an oxygen atom (i.e., —O-alkyl), wherein alkyl is as defined above. Specific examples of “alkoxy” include, but are not limited to, methoxy, ethoxy, propoxy, isopropoxy, cyclohexyloxy, and cyclopentyloxy. An alkoxy group can be optionally substituted with one or more appropriate substituents such as halogen, amino, cyano, or hydroxyl.

As used herein, the term “aryl” or “aryl group,” by itself or as part of another substituent (e.g., aryloxy), refers to and includes monocyclic or polycyclic aromatic hydrocarbon radicals, having the number of annular carbon atoms designated (e.g., C₆₋₁₄ means six to fourteen carbons). Particular aryl groups are those having 6 to 14 annular carbon atoms (a “C₆₋₁₄ aryl”). Examples of “C₆₋₁₄ aryl” include, but are not limited to, phenyl, naphthyl, anthracenyl, and the like. In some embodiments, an aryl may contain a single ring (e.g., phenyl). In some embodiments, an aryl may contain multiple (e.g., two or three) rings. In some embodiments, an aryl may contain multiple condensed rings where at least one of the condensed rings is aromatic (e.g., 1,2,3,4-tetrahydronaphthyl and naphthyl).

As used herein, a combined term such as “arylalkyl” denotes a group including aryl and alkyl wherein aryl is a substituent on alkyl.

As used herein, the term “heterocyclyl” or “heterocycle,” by itself or as part of another substituent (e.g., heterocyclyloxy), refers to monocyclic or bicyclic radicals which may be fully saturated, partially saturated, or fully unsaturated or aromatic, having the number of annular carbon atoms designated (e.g., C₃₋₁₀ means three to ten annular carbon atoms) and containing at least one or more of the same or different heteroatoms selected from N, S or O, provided that at least one annular carbon atom is present and two annular oxygen atoms, if present, do not occupy directly neighboring positions. A “heterocyclyl” or “heterocycle” may be a 3 to 15-membered saturated or partially unsaturated ring containing 1 to 4 heteroatoms selected from O, S and N, where the ring may be monocyclic, bicyclic or tricyclic, contain at least one annular carbon atom and 1 to 3 nitrogen atoms, and/or 1 oxygen or sulfur atom or 1 or 2 oxygen and/or sulfur atoms; provided that when more than one annular oxygen atoms are present, they do not occupy directly neighboring positions. Examples of “heterocyclyl” or “heterocycle” include, but are not limited to, 2-oxiranyl, 2-aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolinyl, 4-isoxazolinyl, 5-isoxazolinyl, 3-isothiazolinyl, 4-isothiazolinyl, 5-isothiazolinyl, 3-pyrazolinyl, 4-pyrazolinyl, 5-pyrazolinyl, 2-oxazolinyl, 4-oxazolinyl, 5-oxazolinyl, 2-thiazolinyl, 4-thiazolinyl, 5-thiazolinyl, 2-imidazolinyl, 4-imidazolinyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-thiadiazol-2-yl, 1,3,4-oxadiazol-2-yl, 1,3,4-triazol-2-yl, 2,3-dihydrofuran-2-yl, 2,3-dihydrofuran-3-yl, 2,4-dihydrofuran-2-yl, 2,4-dihydrofuran-3-yl, 2,3-dihydrothiophen-2-yl, 2,3-dihydrothiophen-3-yl, 2,4-dihydrothiophen-2-yl, 2,4-dihydrothiophen-3-yl, 2-pyrrolin-2-yl.

As used herein, the term “heteroaryl,” by itself or as part of another substituent (e.g., heteroaryloxy), refers to aromatic heterocyclyl or heterocycle as defined herein. Examples of “heteroaryl” include, but are not limited to, 2-furanyl, 3-furanyl, thiophen-2-yl, thiophen-3-yl, IH-pyrrol-2-yl, IH-pyrrol-3-yl, isoxazol-3-yl, isoxazol-4-yl, isoxazol-5-yl, isothiazol-3-yl, isothiazol-4-yl, isothiazol-5-yl.

As used herein, the term “hydroxyalkyl” refers to alkyl group with at least one hydroxyl substituents.

As used herein, the term “amine” or “amino” refers to any compound carrying at least one amino group, including primary amine (i.e., —NH₂), secondary amine (i.e., —NHR), tertiary amine (i.e., —NRR′), as well as cyclic amines, wherein each of R and R′ independently is a non-hydrogen substituent such as optionally substituted aryl, heteroaryl, or lower (e.g., C₁₋₆) alkyl defined above. Examples of cyclic amines include, but are not limited to, pyrrolidine, piperidine, 1-azacycloheptane, morpholine, and piperazine.

As used herein, the term “substituted” whether preceded by the term “optionally” or not, refers to replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any even structure can be substituted with more than one substituent selected from a specified group, the substituent can be either the same or different in every position. A ring substituent, such as heterocycloalkyl, can be bound to another ring, such as a cycloalkyl to form a spiro-bicyclic ring system, e.g., both rings share one common atom. As one or ordinary skill in the rat will recognize, combinations of substituents envisioned by this disclosure are those combinations that result in the formation of stable or chemically feasible compounds. For convenience and as commonly understood, the term “optionally substituted” is used interchangeably with the phrase “substituted or unsubstituted” and only applies to the chemical entities that can be substituted with. As describe herein, when the term “optionally substituted” precedes a list, this term refers to all of the subsequent substitutable group sin that list.

As used herein, the term “therapeutically effective amount” means that amount of active compound or pharmaceutical agent that elicits the biological or medicinal response in a tissue, system, animal or human that is being sought by a researcher, veterinarian, medical doctor or other clinician.

As used herein, the term “treatment” or “treating” refers to the treatment of a mammal afflicted with a pathological condition and refers to an effect that alleviates the condition, e.g., by killing the cancerous cells, but also to an effect that results in the inhibition of the progress of the condition, and includes a reduction in the rate of progress, a halt in the rate of progress, amelioration of the condition, and cure of the condition.

As used herein, the term “pharmaceutically acceptable” pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

As used herein, the term “pharmaceutically acceptable salt”—unless otherwise specified—refers to salts which are suitable for use in contact with the tissues of a subject (e.g., human) without excessive adverse effect. In some embodiments, pharmaceutically acceptable salts include salts of a compound of the invention having an acidic group (e.g., potassium salts, sodium salts, magnesium salts, calcium salts) or a basic group (e.g., sulfate, hydrochloride, phosphate, nitrate, carbonate).

As used herein, the term “patient” refers to a mammal which include humans and non-human mammals such as cows.

Unless specifically otherwise defined, all the terms used herein have their common meanings as known to a skilled person in the art.

### Synthesis of Compounds

Set forth below are some exemplary schemes of methods that have been used or can be used for synthesizing the compounds of this invention:

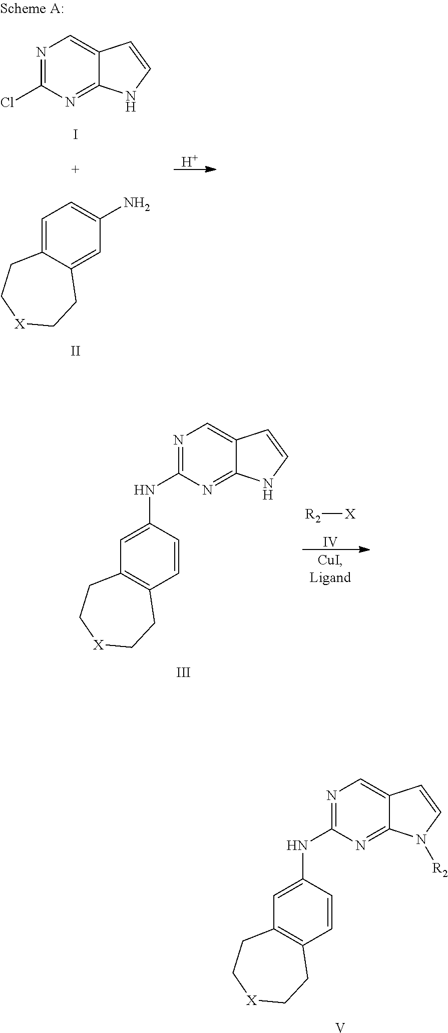

In Scheme A, compound I and Compound II react under an acidic condition to give rise to coupled Compound III which is then treated with Compound IV in the presence of CuI to give Compound V of the invention.

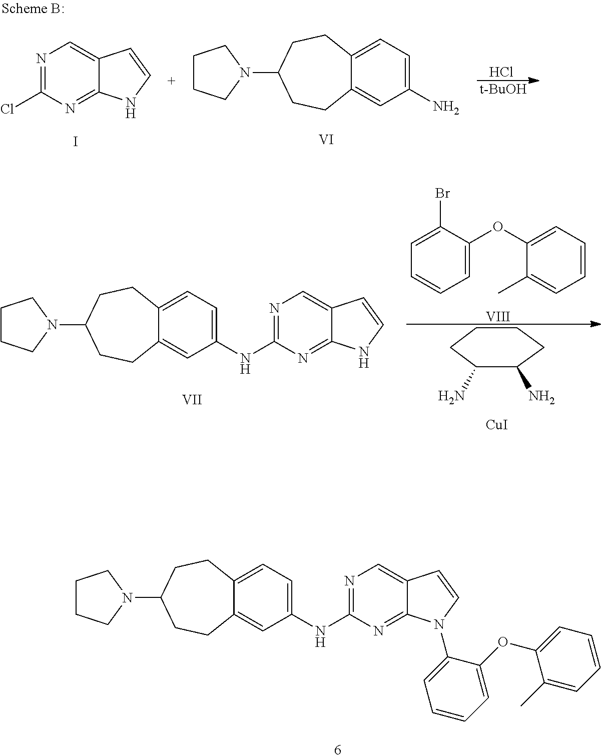

In Scheme B, compound I reacts with compound VI in the presence of an acid (e.g., hydrochloric acid) and alcohol to give rise to compound VII, which then reacts with compound VIII in the presence of a catalyst (e.g., CuI) to provide compound 6.

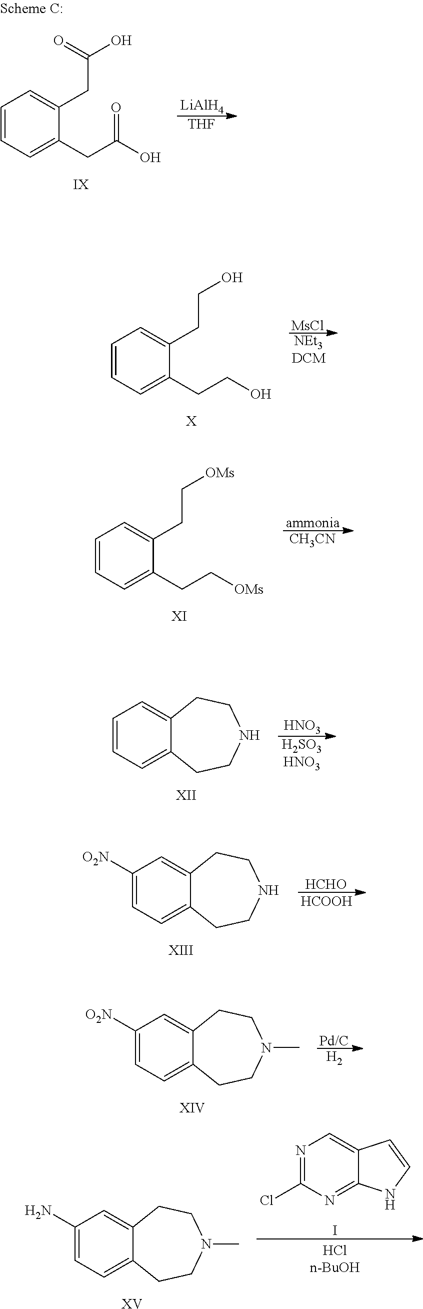

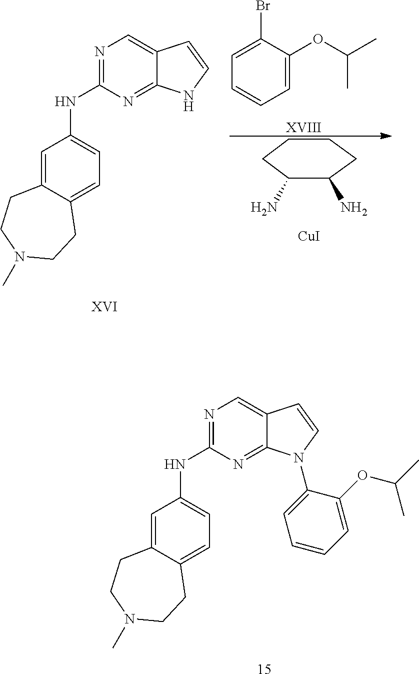

In Scheme C, compound IX reacts with a reducing agent (e.g., LiAlH₄) in an organic solvent (e.g., tetrahydrofuran) to give rise to compound X, which then reacts with MsCl in an organic solvent (e.g., dichloromethane) in the presence of a base (e.g., triethylamine) to provide compound XI. Compound XI reacts with ammonia in the present of CH₃CN to provide compound XII, which then reacts with HNO₃ in the presence of an acid to give compound XIII. Compound XIII then reacts with HCHO in the presence of an acid to provide compound XIV, which in turn is converted to compound XV in the presence of a catalyst and H₂. Compounds IV and I react in the presence of an acid (e.g., hydrochloric acid) and alcohol to give rise to compound XVI, which then reacts with compound XVIII in the presence of a catalyst (e.g., CuI) to provide compound 15.

The present invention is further exemplified by the following examples that illustrate the preparation of the compounds of the invention. These examples are for illustration only, and do not intend to limit to scope of the present invention in any way.

### Example 1: Synthesis of N-(7-(pyrrolidin-1-yl)-6,7,8,9-tetrahydro-5H-benzo[7]annulen-2-yl)-7-(2-(o-tolyloxy)phenyl)-7H-pyrrolo[2,3-d]pyrimidin-2-amine (compound 6)

The title compound was synthesized according to Scheme B with the following detailed.

### Step 1. Preparation of N-(7-(pyrrolidin-1-yl)-6,7,8,9-tetrahydro-5H-benzo[7]annulen-2-yl)-7H-pyrrolo-[2,3-d]pyrimidin-2-amine (VII)

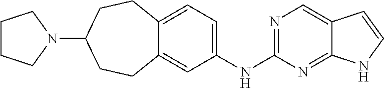 [SMILES: c1cc2cnc(Nc3ccc4c(c3)CCC(N3CCCC3)CC4)nc2[nH]1]

To a solution of 2-chloro-7H-pyrrolo[2,3-d]pyrimidine (280 mg, 1.82 mmol) and 7-(pyrrolidin-1-yl)-6,7,8,9-tetrahydro-5H-benzo[7]annulen-2-amine (461 mg, 2.0 mmol) in n-BuOH (6 mL) was added concentrated hydrochloric acid (0.46 mL, 5.46 mmol). The mixture was stirred in a capped vial at 140° C. overnight, cooled to room temperature, diluted with water (20 mL), basified with 5N NaOH to pH about 10, and extracted with EtOAc (20 mL×3). The organic layer was washed with brine (20 mL), dried over Na₂SO₄, filtered, and concentrated. The residue was purified by column chromatography (DCM:MeOH=30:1 to 8:1, with 1% ammonia) to give the title compound N-(7-(pyrrolidin-1-yl)-6,7,8,9-tetrahydro-5H-benzo[7]annulen-2-yl)-7H-pyrrolo-[2,3-d]pyrimidin-2-amine (485 mg, yield: 76%).

### Step 2. Preparation of N-(7-(pyrrolidin-1-yl)-6,7,8,9-tetrahydro-5H-benzo[7]annulen-2-yl)-7-(2-(o-tolyloxy)phenyl)-7H-pyrrolo[2,3-d]pyrimidin-2-amine (compound 6)

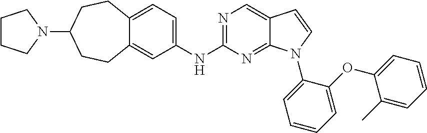 [SMILES: Cc1ccccc1Oc1ccccc1-n1ccc2cnc(Nc3ccc4c(c3)CCC(N3CCCC3)CC4)nc21]

N-(7-(pyrrolidin-1-yl)-6,7,8,9-tetrahydro-5H-benzo[7]annulen-2-yl)-7H-pyrrolo[2,3-d]pyrimidin-2-amine from Step 1 (50 mg, 0.14 mmol), 1-bromo-2-(o-tolyloxy)benzene (37 mg, 0.14 mmol), (trans)-cyclohexane-1,2-diamine (5 mg, 0.042 mmol), CuI (8 mg, 0.042 mmol), and K₃PO₄ (104 mg, 0.49 mmol) in dioxane (2 mL) was stirred at 120° C. under nitrogen overnight. The mixture was diluted with water (5 mL), and extracted with EtOAc (20 mL). The organic layer was washed with brine (10 mL), dried over Na₂SO₄, filtered, and concentrated. The residue was purified by prep-HPLC to give the title compound N-(7-(pyrrolidin-1-yl)-6,7,8,9-tetrahydro-5H-benzo[7]annulen-2-yl)-7-(2-(o-tolyloxy)phenyl)-7H-pyrrolo[2,3-d]pyrimidin-2-amine as a TFA salt (12 mg, yield: 13%).

### Example 2: Synthesis of N-(7-(2-isopropoxyphenyl)-7H-pyrrolo[2,3-d]pyrimidin-2-yl)-3-methyl-2,3,4,5-tetrahydro-1H-benzo[d]azepin-7-amine (compound 15)

The title compound was synthesized according to Scheme C with the following detailed.

### Step 1. Preparation of 2,2′-(1,2-phenylene)diethanol (X)

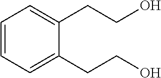 [SMILES: OCCc1ccccc1CCO]

To a solution of 2,2′-(1,2-phenylene)diacetic acid (4.0 g, 20.6 mmol) in THF (80 mL), cooled to 0° C., was added powered LiAlH₄ (3.12 g, 82.4 mmol) slowly. After addition was complete, the mixture was stirred at room temperature overnight. The reaction mixture was quenched with water at −20° C., acidified to pH about 1 to 2 with concentrated hydrochloric acid, and diluted with Et₂O (100 mL). The organic phase was collected, washed with brine (30 mL×3), dried with Na₂SO₄, filtered, and concentrated to give the crude title compound 2,2′-(1,2-phenylene)diethanol (3.0 g, yield: 71%).

### Step 2. Preparation of 1,2-phenylenebis(ethane-2,1-diyl) dimethanesulfonate (XI)

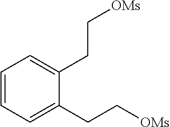 [SMILES: CS(=O)(=O)OCCc1ccccc1CCOS(C)(=O)=O]

2,2′-(1,2-phenylene)diethanol prepared in Step 1 was used without any further purification. To a solution of 2,2′-(1,2-phenylene)diethanol X (3.0 g, 18 mmol) in DCM (30 mL), cooled to 0° C., was added triethylamine (5.46 g, 54 mmol), followed by the addition of methanesulfonyl chloride (6.18 g, 54 mmol). The mixture was stirred at this temperature for 0.5 hour, and diluted with 1N hydrochloric acid (50 mL). The organic phase was separated, washed with brine (10 mL), dried over Na₂SO₄, filtered, and concentrated to give the crude title compound 1,2-phenylenebis(ethane-2,1-diyl) dimethanesulfonate (5.5 g, yield: 94%).

### Step 3. Preparation of 2,3,4,5-tetrahydro-1H-benzo[d]azepine (XII)

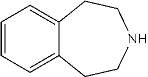 [SMILES: c1ccc2c(c1)CCNCC2]

A solution of 1,2-phenylenebis(ethane-2,1-diyl) dimethanesulfonate from Step 2 (5.5 g, 17 mmol) in CH₃CN (75 mL) and ammonia (28%, 75 mL) was stirred at 100° C. for one hour in an autoclave (pressure raised to ca 40 psi). After cooled to room temperature, the mixture was poured into water (100 mL), and acidified to pH about 4 with concentrated hydrochloric acid. The resultant mixture was extracted with ether (100 mL). The aqueous phase was basified to PH about 14 with 30% NaOH, and extracted with 10% MeOH/DCM (100 mL). The organic phase was dried over Na₂SO₄, filtered, and concentrated in vacuo. The residue was purified by column chromatography (MeOH:DCM=1:50 to 1:10) to give the title compound 2,3,4,5-tetrahydro-1H-benzo[d]azepine (500 mg, yield: 20%).

### Step 4. Preparation of 7-nitro-2,3,4,5-tetrahydro-1H-benzo[d]azepine (XIII)

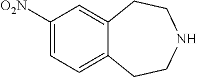 [SMILES: O=[N+]([O-])c1ccc2c(c1)CCNCC2]

To a solution of 2,3,4,5-tetrahydro-1H-benzo[d]azepine from Step 3 (300 mg, 2.04 mmol) in TFA (1.86 g, 16.3 mmol) and concentrated H₂SO₄ (800 mg, 8.2 mmol), cooled to 0° C., was added 65% HNO₃ (217 mg, 3.45 mmol) dropwise. The mixture was stirred at this temperature for 2 hours, poured into ice-water (10 mL), basified to pH about 10 with 5N NaOH, and extracted with EtOAc (50 mL). The organic layer was washed brine (30 mL), dried over Na₂SO₄, filtered, and concentrated to give the title compound 7-nitro-2,3,4,5-tetrahydro-1H-benzo[d]azepine (250 mg, yield: 63%).

### Step 5. Preparation of 3-methyl-7-nitro-2,3,4,5-tetrahydro-1H-benzo[d]azepine (XIV)

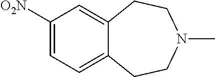

7-nitro-2,3,4,5-tetrahydro-1H-benzo[d]azepine prepared in Step 4 was used without any further purification. A solution of 7-nitro-2,3,4,5-tetrahydro-1H-benzo[d]azepine from Step 4 (250 mg, 1.3 mmol) in 37% formaldehyde (0.8 mL) and 88% formic acid (0.49 mL) was stirred at room temperature for 1 hour, warmed to 70° C., and stirred overnight. The reaction mixture was allowed to cool to room temperature, basified with saturated aqueous NaHCO₃ to pH about 9, and extracted with MTBE (50 mL). The organic layer was washed with brine (30 mL), dried over Na₂SO₄, filtered, and concentrated to give the title compound 3-methyl-7-nitro-2,3,4,5-tetrahydro-1H-benzo[d]azepine as a yellow oil (217 mg, yield: 81%).

### Step 6. Preparation of 3-methyl-2,3,4,5-tetrahydro-1H-benzo[d]azepin-7-amine (XV)

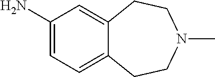

To a solution of 3-methyl-7-nitro-2,3,4,5-tetrahydro-1H-benzo[d]azepine from Step 5 (150 mg, 0.73 mmol) in MeOH (5 mL) was added Pd/C (50 mg, 10% Pd, wet, with 50% water). The mixture was stirred at room temperature under hydrogen atmosphere (45 psi) overnight. The reaction mixture was filtered through celite to remove the catalyst. The filtrated was concentrated. The residue was purified by prep-TLC to give the title compound 3-methyl-2,3,4,5-tetrahydro-1H-benzo[d]azepin-7-amine (48 mg, yield: 39%).

### Step 7. Preparation of 3-methyl-N-(7H-pyrrolo[2,3-d]pyrimidin-2-yl)-2,3,4,5-tetrahydro-1H-benzo[d]-azepin-7-amine (XVI)

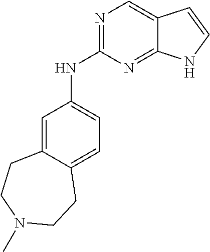 [SMILES: CN1CCc2ccc(Nc3ncc4cc[nH]c4n3)cc2CC1]

To a solution of 2-chloro-7H-pyrrolo[2,3-d]pyrimidine I (40 mg, 0.26 mmol) and 3-methyl-2,3,4,5-tetrahydro-1H-benzo[d]azepin-7-amine from Step 6 (48 mg, 0.28 mmol) in n-BuOH (1 mL) was added concentrated hydrochloric acid (0.065 mL, 0.78 mmol). The mixture was stirred in a capped vial at 140° C. overnight, cooled to room temperature, diluted with water (5 mL), basified with 5N NaOH to pH about 10, and extracted with EtOAc (20 mL). The organic layer was washed with brine (10 mL), dried over Na₂SO₄, filtered, and concentrated to give the title compound 3-methyl-N-(7H-pyrrolo[2,3-d]pyrimidin-2-yl)-2,3,4,5-tetrahydro-1H-benzo[d]-azepin-7-amine (65 mg, crude).

### Step 8. Preparation of N-(7-(2-isopropoxyphenyl)-7H-pyrrolo[2,3-d]pyrimidin-2-yl)-3-methyl-2,3,4,5-tetrahydro-1H-benzo[d]azepin-7-amine (compound 15)

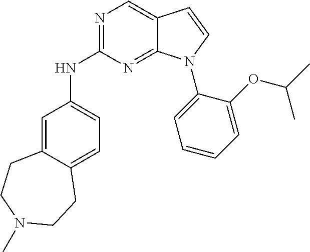 [SMILES: CC(C)Oc1ccccc1-n1ccc2cnc(Nc3ccc4c(c3)CCN(C)CC4)nc21]

3-methyl-N-(7H-pyrrolo[2,3-d]pyrimidin-2-yl)-2,3,4,5-tetrahydro-1H-benzo[d]-azepin-7-amine prepared in Step 7 was used without further purification. A solution of 3-methyl-N-(7H-pyrrolo[2,3-d]pyrimidin-2-yl)-2,3,4,5-tetrahydro-1H-benzo[d]azepin-7-amine from Step 7 (65 mg, crude, about 0.22 mmol), 1-bromo-2-isopropoxybenzene (57 mg, 0.27 mmol), (trans)-cyclohexane-1,2-diamine (7.5 mg, 0.066 mmol), CuI (12 mg, 0.066 mmol), and K₃PO₄ (164 mg, 0.77 mmol) in dioxane (2 mL) was stirred at 120° C. under nitrogen overnight. The mixture was diluted with water (5 mL), and extracted with EtOAc (20 mL). The organic layer was washed with brine (10 mL), dried over Na₂SO₄, filtered, and concentrated. The residue was purified by prep-TLC to give the title compound N-(7-(2-isopropoxyphenyl)-7H-pyrrolo[2,3-d]pyrimidin-2-yl)-3-methyl-2,3,4,5-tetrahydro-1H-benzo[d]azepin-7-amine (9 mg, yield: 9%).

Table 1 below lists exemplary compounds of this invention that were synthesized largely according to Scheme A, B, or C. For instance, Compound Nos. 1 and 3-14 were prepared according to Scheme A or B and compound 15 was prepared according to Scheme C.

TABLE 1

| Compound | | | |
|---|---|---|---|
| No. | Chemical Name | LCMS (M + H) | NMR |
| 1 | 7-(2- | 482.3 found | ¹H NMR (400 MHz, CDCl₃) δ 8.67 (s, |
| | isopropoxyphenyl)- | 482.3 required | 1H), 7.66 (s, 2H), 7.37 (d, J = 15.0 Hz, |
| | N-(7-(pyrrolidin-1- | | 2H), 7.24 (s, 1H), 7.10 (d, J = 7.7 Hz, |
| | yl)-6,7,8,9- | | 2H), 6.97 (d, J = 8.0 Hz, 1H), |
| | tetrahydro-5H- | | 6.50 (d, J = 3.6 Hz, 1H), 4.45 (m, |
| | benzo[7]annulen-2- | | 1H), 3.48 (m, 1H), 3.39 (m, 2H), |
| | yl)-7H-pyrrolo[2,3- | | 2.81 (m, 2H), 2.74 (m, 2H), 2.52 (m, |
| | d]pyrimidin-2-amine | | 2H), 2.06 (m, 6H), 1.55 (m, 2H), |
| | | | 1.20 (d, J = 6.1 Hz, 6H). |
| 2 | 7-(2- | 432.0 found | ¹H NMR (400 MHz, CDCl₃) δ 8.64 (s, |
| | isopropoxyphenyl)- | 432.3 required | 1H), 7.97 (s, 1H), 7.61 (d, J = 7.1 Hz, |
| | N-(1-(1- | | 1H), 7.41 (d, J = 21.3 Hz, 3H), |
| | methylpiperidin-4- | | 7.18 (d, J = 3.4 Hz, 1H), 7.11 (d, J = 8.0 Hz, |
| | yl)-1H-pyrazol-4-yl)- | | 2H), 6.49 (d, J = 3.5 Hz, 1H), |
| | 7H-pyrrolo[2,3- | | 4.52-4.37 (m, 1H), 4.17 (bs, 1H), |
| | d]pyrimidin-2-amine | | 3.15 (s, 2H), 2.70 (bs, 1H), 2.55 (bs, |
| | | | 3H), 2.40 (bs, 2H), 2.17 (bs, 2H), |
| | | | 1.19 (d, J = 5.9 Hz, 6H). |
| 3 | 7-(3- | 482.3 found | ¹H NMR (400 MHz, CDCl₃) δ |
| | isopropoxyphenyl)- | 482.3 required | 12.33 (s, 1H), 8.69 (s, 1H), 7.57 (s, 1H), |
| | N-(7-(pyrrolidin-1- | | 7.45 (d, J = 8.1 Hz, 1H), 7.39 (s, 1H), |
| | yl)-6,7,8,9- | | 7.29 (s, 1H), 7.25 (s, 1H), 7.18 (s, |
| | tetrahydro-5H- | | 1H), 7.05 (d, J = 8.2 Hz, 1H), 6.90 (d, |
| | benzo[7]annulen-2- | | J = 7.0 Hz, 1H), 6.56 (d, J = 3.7 Hz, |
| | yl)-7H-pyrrolo[2,3- | | 1H), 4.68-4.50 (m, 1H), 3.63 (s, |
| | d]pyrimidin-2-amine | | 2H), 3.40 (s, 1H), 3.00-2.83 (m, |
| | | | 2H), 2.82-2.62 (m, 2H), 2.52 (s, |
| | | | 2H), 2.20 (s, 2H), 1.95 (s, 2H), |
| | | | 1.61 (m, 4H), 1.37 (d, J = 5.9 Hz, 6H). |
| 4 | 7-(2- | 522.4 found | ¹H NMR (400 MHz, DMSO) δ |
| | (cyclohexyloxy)phenyl)- | 522.3 required | 10.51 (s, 1H), 9.27 (s, 1H), 8.74 (s, 1H), |
| | N-(7-(pyrrolidin- | | 7.74 (s, 1H), 7.58 (d, J = 7.4 Hz, 1H), |
| | 1-yl)-6,7,8,9- | | 7.43 (s, 1H), 7.39-7.26 (m, 3H), |
| | tetrahydro-5H- | | 7.12 (s, 1H), 6.95 (d, J = 7.9 Hz, 1H), |
| | benzo[7]annulen-2- | | 6.59 (s, 1H), 4.45-4.28 (m, 1H), |
| | yl)-7H-pyrrolo[2,3- | | 3.59-3.35 (m, 4H), 3.17-2.99 (m, |
| | d]pyrimidin-2-amine | | 2H), 2.80-2.57 (m, 4H), |
| | | | 2.40-2.22 (m, 2H), 1.89 (s, 4H), 1.72 (s, |
| | | | 2H), 1.53-1.26 (m, 8H). |
| 5 | 7-(2- | 516.3 found | ¹H NMR (400 MHz, CDCl₃) δ |
| | phenoxyphenyl)-N- | 516.3 required | 12.48 (s, 1H), 11.71 (s, 1H), 8.42 (s, 1H), |
| | (7-(pyrrolidin-1-yl)- | | 7.70 (d, J = 2.1 Hz, 1H), 7.65 (dd, J = 7.9, |
| | 6,7,8,9-tetrahydro- | | 1.5 Hz, 1H), 7.52 (d, J = 7.5 Hz, |
| | 5H- | | 1H), 7.48 (dd, J = 8.1, 2.2 Hz, 1H), |
| | benzo[7]annulen-2- | | 7.41 (d, J = 3.9 Hz, 1H), 7.35 (dd, J = 7.7, |
| | yl)-7H-pyrrolo[2,3- | | 1.1 Hz, 1H), 7.25-7.19 (m, |
| | d]pyrimidin-2-amine | | 2H), 7.14 (dd, J = 8.3, 1.0 Hz, 1H), |
| | | | 7.10-7.04 (m, 2H), 6.88-6.84 (m, |
| | | | 2H), 6.64 (d, J = 3.9 Hz, 1H), 3.71 (s, |
| | | | 2H), 3.46 (s, 1H), 2.90 (dd, J = 14.8, |
| | | | 6.6 Hz, 4H), 2.76-2.69 (m, 2H), |
| | | | 2.36 (s, 2H), 2.15 (m, 2H), 1.99 (s, |
| | | | 2H), 1.52 (m, 2H). |
| 6 | N-(7-(pyrrolidin-1- | 530.3 found | ¹H NMR (400 MHz, CDCl₃) δ |
| | yl)-6,7,8,9- | 530.3 required | 12.20 (s, 1H), 11.70 (s, 1H), 8.46 (s, 1H), |
| | tetrahydro-5H- | | 7.77-7.63 (m, 2H), 7.58-7.43 (m, |
| | benzo[7]annulen-2- | | 3H), 7.31 (s, 1H), 7.20 (d, J = 3.4 Hz, |
| | yl)-7-(2-(o- | | 1H), 7.04 (dd, J = 4.7, 2.4 Hz, 3H), |
| | tolyloxy)phenyl)-7H- | | 6.94 (d, J = 8.3 Hz, 1H), |
| | pyrrolo[2,3- | | 6.84-6.75 (m, 1H), 6.68 (d, J = 3.9 Hz, 1H), |
| | d]pyrimidin-2-amine | | 3.72 (s, 2H), 3.47 (s, 1H), 2.92 (s, |
| | | | 4H), 2.75 (m, 2H), 2.37 (s, 2H), |
| | | | 2.17 (m, 2H), 2.09 (s, 3H), 2.00 (s, 2H), |
| | | | 1.52 (m, 2H). |
| 7 | 7-(4- | 482.4 found | ¹H NMR (400 MHz, CDCl₃) δ 8.70 (s, |
| | isopropoxyphenyl)- | 482.3 required | 1H), 7.65 (d, J = 8.9 Hz, 2H), 7.59 (d, |
| | N-(7-(pyrrolidin-1- | | J = 1.8 Hz, 1H), 7.44 (dd, J = 8.1, 2.0 Hz, |
| | yl)-6,7,8,9- | | 2H), 7.24 (d, J = 3.7 Hz, 1H), |
| | tetrahydro-5H- | | 7.05 (dd, J = 8.6, 3.3 Hz, 3H), |
| | benzo[7]annulen-2- | | 6.58 (d, J = 3.7 Hz, 1H), 4.70-4.59 (m, |
| | yl)-7H-pyrrolo[2,3- | | 1H), 3.68 (m, 2H), 3.42 (m, 1H), |
| | d]pyrimidin-2-amine | | 2.97-2.84 (m, 4H), 2.77 (m, 2H), |
| | | | 2.56 (m, 2H), 2.23 (m, 2H), 1.98 (m, |
| | | | 2H), 1.68-1.57 (m, 2H), 1.43 (d, J = 6.0 Hz, |
| | | | 6H). |
| 8 | 7-([1,1′-biphenyl]-4- | 500.4 found | ¹H NMR (400 MHz, CDCl₃) δ 8.72 (s, |
| | yl)-N-(7-(pyrrolidin- | 500.3 required | 1H), 7.87 (d, J = 8.7 Hz, 1H), 7.75 (d, |
| | 1-yl)-6,7,8,9- | | J = 8.7 Hz, 1H), 7.65 (s, 2H), 7.51 (s, |
| | tetrahydro-5H- | | 1H), 7.40 (s, 1H), 7.32 (s, 1H), |
| | benzo[7]annulen-2- | | 7.25-7.19 (m, 1H), 7.05 (d, J = 7.5 Hz, |
| | yl)-7H-pyrrolo[2,3- | | 1H), 6.61 (s, 1H), 3.72-3.44 (m, |
| | d]pyrimidin-2-amine | | 2H), 3.39-3.30 (m, 1H), |
| | | | 2.95-2.67 (m, 4H), 2.54 (s, 1H), 2.41 (s, |
| | | | 1H), 2.24-1.82 (m, 4H), |
| | | | 1.72-1.51 (m, 4H). |
| 9 | 7-(2-isopropoxy-4- | 512.4 found | ¹H NMR (400 MHz, CDCl₃) δ 8.64 (s, |
| | methoxyphenyl)-N- | 512.3 required | 1H), 7.62 (s, 1H), 7.49 (d, J = 8.2 Hz, |
| | (7-(pyrrolidin-1-yl)- | | 1H), 7.41 (s, 1H), 7.30 (s, 1H), |
| | 6,7,8,9-tetrahydro- | | 7.15 (s, 1H), 6.98 (d, J = 7.6 Hz, 1H), |
| | 5H- | | 6.64 (s, 2H), 6.49 (s, 1H), 4.41 (m, 1H), |
| | benzo[7]annulen-2- | | 4.12 (m, 1H), 3.89 (s, 3H), 3.39 (m, |
| | yl)-7H-pyrrolo[2,3- | | 2H), 2.78 (m, 4H), 2.50 (m, 2H), |
| | d]pyrimidin-2-amine | | 2.04 (s, 6H), 1.56 (m, 2H), |
| | | | 1.19 (d, J = 5.9 Hz, 6H). |
| 10 | 7-(4-chloro-2- | 516.3 found | ¹H NMR (400 MHz, CDCl₃) δ 8.66 (s, |
| | isopropoxyphenyl)- | 516.3 required | 1H), 7.60 (s, 2H), 7.40 (s, 1H), |
| | N-(7-(pyrrolidin-1- | | 7.27 (s, 1H), 7.18 (d, J = 3.6 Hz, 1H), |
| | yl)-6,7,8,9- | | 7.14-6.87 (m, 3H), 6.51 (d, J = 3.7 Hz, |
| | tetrahydro-5H- | | 1H), 4.47 (m, 1H), 3.70 (m, 2H), |
| | benzo[7]annulen-2- | | 3.44 (m, 1H), 2.83 (m, 2H), 2.76 (m, |
| | yl)-7H-pyrrolo[2,3- | | 2H), 2.57 (m, 2H), 2.19 (m, 2H), |
| | d]pyrimidin-2-amine | | 2.04 (m, 4H), 1.60 (m, 2H), 1.24 (d, |
| | | | J = 5.8 Hz, 6H). |
| 11 | N-isopropyl-2-(2-((7- | 509.6 found | ¹H NMR (400 MHz, CDCl₃) δ 8.70 (s, |
| | (pyrrolidin-1-yl)- | 509.3 required | 1H), 7.79 (d, J = 6.7 Hz, 1H), 7.55 (d, |
| | 6,7,8,9-tetrahydro- | | J = 18.3 Hz, 3H), 7.46 (s, 1H), |
| | 5H- | | 7.34 (s, 1H), 7.21 (d, J = 7.7 Hz, 1H), |
| | benzo[7]annulen-2- | | 7.11 (s, 1H), 6.97 (d, J = 7.9 Hz, 1H), |
| | yl)amino)-7H- | | 6.59 (s, 1H), 3.90 (m, 1H), 3.35 (m, 2H), |
| | pyrrolo[2,3- | | 2.81 (m, 1H), 2.78-2.62 (m, 4H), |
| | d]pyrimidin-7- | | 2.48 (m, 2H), 2.06 (s, 6H), |
| | yl)benzamide | | 1.59-1.49 (m, 2H), 1.25 (d, 6H). |
| 12 | 7-(2′-methyl-[1,1′- | 514.4 found | ¹H NMR (400 MHz, CDCl₃) δ 8.73 (s, |
| | biphenyl]-4-yl)-N-(7- | 514.3 required | 1H), 7.86 (d, J = 8.4 Hz, 2H), |
| | (pyrrolidin-1-yl)- | | 7.68-7.59 (m, 1H), 7.48 (d, J = 8.4 Hz, |
| | 6,7,8,9-tetrahydro- | | 2H), 7.40 (d, J = 7.6 Hz, 1H), |
| | 5H- | | 7.36-7.27 (m, 4H), 7.18 (s, 1H), 7.03 |
| | benzo[7]annulen-2- | | (d, J = 7.9 Hz, 1H), 6.60 (d, J = 3.6 Hz, |
| | yl)-7H-pyrrolo[2,3- | | 1H), 3.40-2.99 (m, 3H), |
| | d]pyrimidin-2-amine | | 2.96-2.64 (m, 4H), 2.56-2.43 (m, 1H), |
| | | | 2.36 (b, 4H), 2.11-1.89 (m, 6H), |
| | | | 1.73-1.46 (m, 2H) |
| 13 | 7-(3- | 483.5 found | ¹H NMR (400 MHz, CDCl₃) δ 8.67 (s, |
| | isopropoxypyridin-2- | 483.3 required | 1H), 8.25 (s, 1H), 7.64 (s, 1H), |
| | yl)-N-(7-(pyrrolidin- | | 7.50-7.37 (m, 2H), 7.28 (d, J = 3.7 Hz, |
| | 1-yl)-6,7,8,9- | | 2H), 6.97 (d, J = 7.8 Hz, 1H), 6.55 (s, |
| | tetrahydro-5H- | | 1H), 4.49 (s, 1H), 3.64 (s, 1H), |
| | benzo[7]annulen-2- | | 3.46 (m, 2H), 2.91-2.66 (m, 4H), |
| | yl)-7H-pyrrolo[2,3- | | 2.59 (s, 1H), 2.46 (s, 1H), 2.09 (m, 6H), |
| | d]pyrimidin-2-amine | | 1.52 (m, 2H), 1.21 (d, J = 6.0 Hz, |
| | | | 6H). |
| 14 | 7-(3-isopropoxy- | 558.4 found | ¹H NMR (400 MHz, CDCl₃) δ 8.69 (s, |
| | [1,1′-biphenyl]-4-yl)- | 558.3 required | 1H), 7.73 (s, 1H), 7.65 (d, J = 6.9 Hz, |
| | N-(7-(pyrrolidin-1- | | 3H), 7.51 (s, 2H), 7.40 (s, 1H), |
| | yl)-6,7,8,9- | | 7.26 (s, 5H), 6.99 (s, 1H), 6.54 (s, 1H), |
| | tetrahydro-5H- | | 4.54 (m, 1H), 3.22 (m, 2H), |
| | benzo[7]annulen-2- | | 3.10-3.04 (m, 1H), 2.74 (m, 4H), 2.44 (m, |
| | yl)-7H-pyrrolo[2,3- | | 2H), 2.23 (m, 2H), 1.97 (m, 4H), |
| | d]pyrimidin-2-amine | | 1.50 (m, 2H), 1.26 (d, J = 6.0 Hz, 6H) |
| 15 | N-(7-(2- | 428.1 found | ¹H NMR (400 MHz, CDCl₃) δ 8.67 (s, |
| | isopropoxyphenyl)- | 428.2 required | 1H), 7.83 (s, 1H), 7.62 (s, 1H), |
| | 7H-pyrrolo[2,3- | | 7.40 (d, J = 18.6 Hz, 2H), 7.24 (s, 2H), |
| | d]pyrimidin-2-yl)-3- | | 7.10 (d, J = 7.6 Hz, 2H), 6.99 (d, J = 8.1 Hz, |
| | methyl-2,3,4,5- | | 1H), 6.52 (s, 1H), 4.46 (m, |
| | tetrahydro-1H- | | 1H), 3.17 (b, 8H), 2.79 (s, 3H), |
| | benzo[d]azepin-7- | | 1.20 (d, J = 5.5 Hz, 6H). |
| | amine | | |

### Example 3: AXL Enzymatic Assay

Compounds were tested in a LanthaScreen™ time-resolved fluorescence energy transfer (TR-FRET) enzymatic assay from Invitrogen. The assay used human AXL (Invitrogen, Cat. PV3971) recombinantly expressed his-tagged catalytic domain (amino acids 473-894) from insect cells. The substrate was fluorescein-labeled Poly GT (Invitrogen, Cat. PV3610). Test compounds were prepared and diluted in DMSO in 3-fold serial dilutions to 100× of the final testing concentrations. The compounds were then further diluted to 4× by the kinase reaction buffer (Invitrogen, Cat. PV3189). The enzymatic reaction for compound testing was performed in a white 384-well polypropylene plate (Packard, Cat. 6005214) with a total reaction volume of 10 μl containing 200 ng/ml AXL, 200 nM substrate, and 18 μM ATP that is around its Kₘ. The assay started with loading 2.5 μl of AXL diluted in kinase reaction buffer to wells, followed by addition of equal volume of 4× compounds for 15-min incubation at the room temperature for pre-treatment. The enzymatic reaction was initiated by addition of 5 μl of mixture of the substrate and ATP prepared in kinase reaction buffer. After one hour reaction, 10 μl mixture of EDTA (final 10 mM) and terbium-labeled anti-PY20 antibody (final 2 nM) (Invitrogen, Cat. PV3552) prepared in TR-FRET antibody dilution buffer (Invitrogen, Cat. PV3574) was added to stop the enzymatic reaction and produce TR-FRET signals. After 30 minutes of incubation at room temperature, the plate was read in Tecan Infinite F200 Pro with the following settings: Excitation 340 nm (30)/Emission1 495 nm (10)/Emission2 520 nm (25). The TR-FRET values were dimensionless numbers that were calculated as the ratio of the acceptor (Green Fluorescent Protein) signal to the donor (Terbium) signal. Percent of control was calculated as the percentage of compound-treated vs 1% DMSO vehicle-treated. The dose-response curves were generated and the IC₅₀s were calculated by nonlinear sigmoid curve fitting using GraphPad Prism.

The growth inhibition activities of the disclosed compounds are shown in Table 2 below. In this table, the letter “A” represents an IC₅₀ value within the range of 0 to 25 (including 25) nM; the letter “B” represents an IC₅₀ value within the range of 25 to 50 (including 50) nM; the letter “C” represents an IC₅₀ value within the range of 50 to 200 (including 200) nM; the letter “D” represents an IC₅₀ value within the range of 200 to 400 (including 400) nM; the letter “E” represents an IC₅₀ value within the range of 700 to 800 (including 800) nM; and the letter “F” represents an IC₅₀ value within the range of over 1000 Nm.

TABLE 2
AXL IC₅₀ Data

| Compound No. | Compound Name | AXL IC₅₀ |
|---|---|---|
| 1 | 7-(2-isopropoxyphenyl)-N-(7-(pyrrolidin-1-yl)-6,7,8,9- | B |
| | tetrahydro-5H-benzo[7]annulen-2-yl)-7H-pyrrolo[2,3- | |
| | d]pyrimidin-2-amine | |
| 2 | 7-(2-isopropoxyphenyl)-N-(1-(1-methylpiperidin-4-yl)-1H- | E |
| | pyrazol-4-yl)-7H-pyrrolo[2,3-d]pyrimidin-2-amine | |
| 3 | 7-(3-isopropoxyphenyl)-N-(7-(pyrrolidin-1-yl)-6,7,8,9- | C |
| | tetrahydro-5H-benzo[7]annulen-2-yl)-7H-pyrrolo[2,3- | |
| | d]pyrimidin-2-amine | |
| 4 | 7-(2-(cyclohexyloxy)phenyl)-N-(7-(pyrrolidin-1-yl)-6,7,8,9- | D |
| | tetrahydro-5H-benzo[7]annulen-2-yl)-7H-pyrrolo[2,3- | |
| | d]pyrimidin-2-amine | |
| 5 | 7-(2-phenoxyphenyl)-N-(7-(pyrrolidin-1-yl)-6,7,8,9- | E |
| | tetrahydro-5H-benzo[7]annulen-2-yl)-7H-pyrrolo[2,3- | |
| | d]pyrimidin-2-amine | |
| 6 | N-(7-(pyrrolidin-1-yl)-6,7,8,9-tetrahydro-5H- | A |
| | benzo[7]annulen-2-yl)-7-(2-(o-tolyloxy)phenyl)-7H- | |
| | pyrrolo[2,3-d]pyrimidin-2-amine | |
| 7 | 7-(4-isopropoxyphenyl)-N-(7-(pyrrolidin-1-yl)-6,7,8,9- | B |
| | tetrahydro-5H-benzo[7]annulen-2-yl)-7H-pyrrolo[2,3- | |
| | d]pyrimidin-2-amine | |
| 8 | 7-([1,1′-biphenyl]-4-yl)-N-(7-(pyrrolidin-1-yl)-6,7,8,9- | F |
| | tetrahydro-5H-benzo[7]annulen-2-yl)-7H-pyrrolo[2,3- | |
| | d]pyrimidin-2-amine | |
| 9 | 7-(2-isopropoxy-4-methoxyphenyl)-N-(7-(pyrrolidin-1-yl)- | C |
| | 6,7,8,9-tetrahydro-5H-benzo[7]annulen-2-yl)-7H- | |
| | pyrrolo[2,3-d]pyrimidin-2-amine | |
| 10 | 7-(4-chloro-2-isopropoxyphenyl)-N-(7-(pyrrolidin-1-yl)- | A |
| | 6,7,8,9-tetrahydro-5H-benzo[7]annulen-2-yl)-7H- | |
| | pyrrolo[2,3-d]pyrimidin-2-amine | |
| 11 | N-isopropyl-2-(2-((7-(pyrrolidin-1-yl)-6,7,8,9-tetrahydro-5H- | F |
| | benzo[7]annulen-2-yl)amino)-7H-pyrrolo[2,3-d]pyrimidin-7- | |
| | yl)benzamide | |
| 12 | 7-(2′-methyl-[1,1′-biphenyl]-4-yl)-N-(7-(pyrrolidin-1-yl)- | B |
| | 6,7,8,9-tetrahydro-5H-benzo[7]annulen-2-yl)-7H- | |
| | pyrrolo[2,3-d]pyrimidin-2-amine | |
| 13 | 7-(3-isopropoxypyridin-2-yl)-N-(7-(pyrrolidin-1-yl)-6,7,8,9- | D |
| | tetrahydro-5H-benzo[7]annulen-2-yl)-7H-pyrrolo[2,3- | |
| | d]pyrimidin-2-amine | |
| 14 | 7-(3-isopropoxy-[1,1′-biphenyl]-4-yl)-N-(7-(pyrrolidin-1-yl)- | D |
| | 6,7,8,9-tetrahydro-5H-benzo[7]annulen-2-yl)-7H- | |
| | pyrrolo[2,3-d]pyrimidin-2-amine | |
| 15 | N-(7-(2-isopropoxyphenyl)-7H-pyrrolo[2,3-d]pyrimidin-2-yl)- | D |
| | 3-methyl-2,3,4,5-tetrahydro-1H-benzo[d]azepin-7-amine | |

1. A compound of Formula I 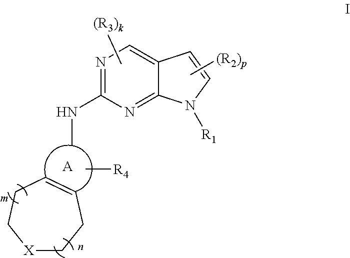
or a pharmaceutically acceptable salt thereof, wherein:
A is a 5- or 6-membered aryl or heteroaryl, and is optionally substituted with one or more R₄ groups;
p is 0, 1, or 2; k is 0 or 1;
each of m and n independently is 0, 1, 2, or 3, and the sum of m and n is less than 4;
X is CHR₅ or NR₆;
R₁ is hydrogen, aryl, heteroaryl, cycloalkyl, or heterocyclyl, and is optionally substituted with 1 to 4 Rₐ groups;
each of R₂ and R₃ independently is halogen, alkyl, alkenyl, alkynyl, haloalkyl, hydroxyl, hydroxylalkyl, alkoxy, alkenyloxy, alkynyloxy, carbonyl, carboxyl, cyano, amino, nitrile, sulfonyl, sulfinyl, sulfhydryl, aryl, cycloalkyl, heteroaryl, or heterocyclyl;
each optional R₄ group independently is halogen, alkyl, alkenyl, alkynyl, haloalkyl, hydroxyl, hydroxylalkyl, alkoxy, alkenyloxy, alkynyloxy, carbonyl, carboxyl, cyano, amino, nitrile, sulfonyl, sulfinyl, sulfhydryl, aryl, cycloalkyl, heteroaryl, or heterocyclyl;
R₅ is hydrogen, amine, alkylamine, cyclic amine, heterocyclyl, alkyl, alkenyl, alkynyl, cycloalkyl, aryl, heteroaryl, nitrile, sulfonyl, sulfinyl, sulfhydryl, halogen, haloalkyl, hydroxyl, hydroxyalkyl, alkoxy, alkenyloxy, alkynyloxy, carbonyl, or carboxyl;
R₆ is hydrogen, alkyl, alkenyl, cycloalkyl, alkynyl, aryl, CN, heteroaryl, or heterocyclyl;
each optional Rₐ group independently is halogen, alkyl, cycloalkyl, alkenyl, alkynyl, aryl, heteroaryl, heterocyclyl, alkoxy, alkenyloxy, alkynyloxy, cycloalkyloxy, aryloxy, heteroaryloxy, heterocyclyloxy, alkylamino, amino carbonyl, acyl, carbonyl, carboxyl, amino, cyano, cyanato, nitrile, sulfonyl, sulfinyl, or sulfhydryl.

2. The compound of claim 1, wherein A is a 6- or 5-membered heteroaryl having 1 to 3 heteroatoms each of which independently is O, S, or N, and A is optionally substituted with 1 to 3 R₄ groups.

3. The compound of claim 1, wherein A is

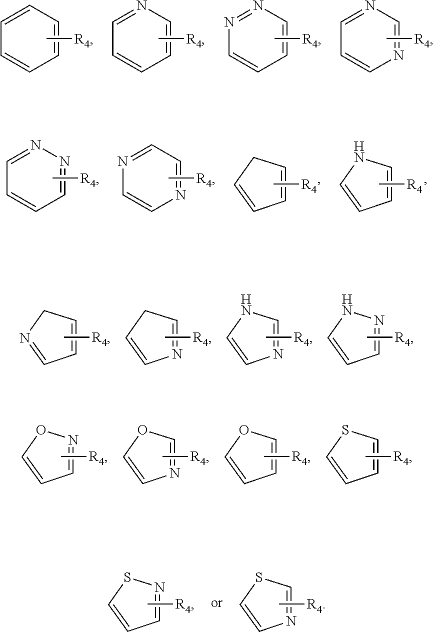

4. The compound of claim 3, wherein A is

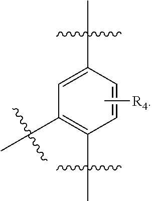

5. The compound of claim 1, wherein R₁ is aryl or heteroaryl and is optionally substituted with 1 to 4 Rₐ groups.

6. The compound of claim 5, wherein R₁ is

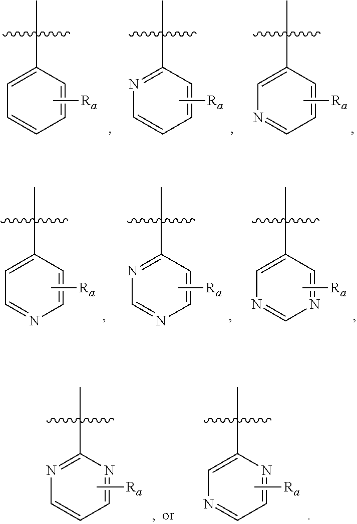

7. The compound of claim 1, wherein each Rₐ independently is halogen, alkyl, aryl, heteroaryl, cycloalkyl, alkoxy, cycloalkyloxy, aryloxy, amino carbonyl, cyano, cyanato, amino, or hydroxyl.

8. The compound of claim 7, wherein Rₐ is isopropoxy, optionally substituted phenyl, or optionally substituted phenoxy.

9. The compound of claim 1, wherein R₅ is 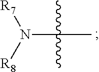
each of R₇ and R₈ independently is hydrogen, alkyl, cycloalkyl, aryl, heteroaryl, cyano, alkoxy, hydroxyl, carbonyl, carboxyl, or hydroxylalkyl; or R₇ and R₈, together with the nitrogen atom to which they are attached, form a 4- to 8-membered heterocyclyl or heteroaryl.

10. The compound of claim 9, wherein R₅ is

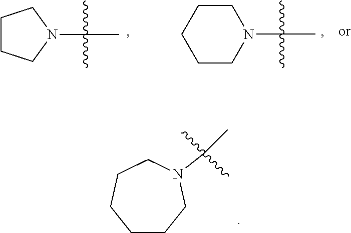 [SMILES: *N1CCCC1 ; *N1CCCCC1 ; *N1CCCCCC1]

11. The compound of claim 1, wherein R₆ is alkyl or cycloalkyl.

12. The compound of claim 1, wherein m is 1 and n is 1.

13. The compound of claim 1, wherein the compound is of Formula II:

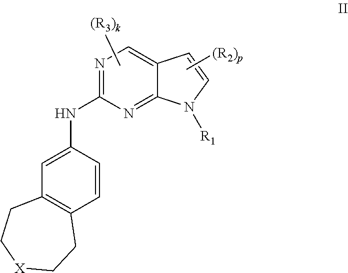

14. The compound of claim 13, wherein k is 0; P is 0; R₁ is 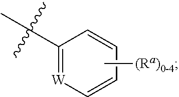
W is CR_{b}, CH, or N; each of Rₐ and R_{b} independently is halogen, alkyl, aryl, heteroaryl, cycloalkyl, alkoxy, cycloalkyloxy, aryloxy, amino carbonyl, cyano, cyanato, amino, or hydroxyl.

15. The compound of claim 14, wherein W is CR_{b}, CH, or N; R_{b} is halogen or lower alkyl; and each Rₐ independently is halogen, aryl, heteroaryl, alkoxy, cycloalkyloxy, or aryloxy.

16. The compound of claim 15, wherein Rₐ is isopropoxy, optionally substituted phenyl, or optionally substituted phenoxy.

17. The compound of any of claim 13, wherein X is CHR₅ or NR₆;
R₅ is 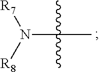
each of R₇ and R₈ independently is hydrogen or alkyl; or R₇ and R₈, together with the nitrogen atom to which they are attached, form a 4- to 8-membered heterocyclyl or heteroaryl; and
R₆ is alkyl or cycloalkyl.

18. The compound of claim 17, wherein R₅ is

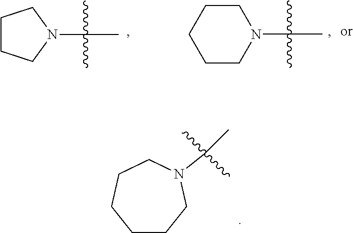 [SMILES: *N1CCCC1 ; *N1CCCCC1 ; *N1CCCCCC1]

19. The compound of claim 1, wherein the compound is
7-(2-isopropoxyphenyl)-N-(7-(pyrrolidin-1-yl)-6,7,8,9-tetrahydro-5H-benzo[7]annulen-2-yl)-7H-pyrrolo[2,3-d]pyrimidin-2-amine;
7-(3-isopropoxyphenyl)-N-(7-(pyrrolidin-1-yl)-6,7,8,9-tetrahydro-5H-benzo[7]annulen-2-yl)-7H-pyrrolo[2,3-d]pyrimidin-2-amine;
7-(4-isopropoxyphenyl)-N-(7-(pyrrolidin-1-yl)-6,7,8,9-tetrahydro-5H-benzo[7]annulen-2-yl)-7H-pyrrolo[2,3-d]pyrimidin-2-amine;
7-(2-phenoxyphenyl)-N-(7-(pyrrolidin-1-yl)-6,7,8,9-tetrahydro-5H-benzo[7]annulen-2-yl)-7H-pyrrolo[2,3-d]pyrimidin-2-amine;
7-(2-(cyclohexyloxy)phenyl)-N-(7-(pyrrolidin-1-yl)-6,7,8,9-tetrahydro-5H-benzo[7]annulen-2-yl)-7H-pyrrolo[2,3-d]pyrimidin-2-amine;
N-(7-(pyrrolidin-1-yl)-6,7,8,9-tetrahydro-5H-benzo[7]annulen-2-yl)-7-(2-(o-tolyloxy)phenyl)-7H-pyrrolo[2,3-d]pyrimidin-2-amine;
N-isopropyl-2-(2-((7-(pyrrolidin-1-yl)-6,7,8,9-tetrahydro-5H-benzo[7]annulen-2-yl)amino)-7H-pyrrolo[2,3-d]pyrimidin-7-yl)benzamide;
7-(4-chloro-2-isopropoxyphenyl)-N-(7-(pyrrolidin-1-yl)-6,7,8,9-tetrahydro-5H-benzo[7]annulen-2-yl)-7H-pyrrolo[2,3-d]pyrimidin-2-amine;
7-(2-isopropoxy-4-methoxyphenyl)-N-(7-(pyrrolidin-1-yl)-6,7,8,9-tetrahydro-5H-benzo[7]annulen-2-yl)-7H-pyrrolo[2,3-d]pyrimidin-2-amine;
7-(3-isopropoxy-[1,1′-biphenyl]-4-yl)-N-(7-(pyrrolidin-1-yl)-6,7,8,9-tetrahydro-5H-benzo[7]annulen-2-yl)-7H-pyrrolo[2,3-d]pyrimidin-2-amine;
7-([1,1′-biphenyl]-4-yl)-N-(7-(pyrrolidin-1-yl)-6,7,8,9-tetrahydro-5H-benzo[7]annulen-2-yl)-7H-pyrrolo[2,3-d]pyrimidin-2-amine;
7-(2′-methyl-[1,1′-biphenyl]-4-yl)-N-(7-(pyrrolidin-1-yl)-6,7,8,9-tetrahydro-5H-benzo[7]annulen-2-yl)-7H-pyrrolo[2,3-d]pyrimidin-2-amine;
7-(3-isopropoxypyridin-2-yl)-N-(7-(pyrrolidin-1-yl)-6,7,8,9-tetrahydro-5H-benzo[7]annulen-2-yl)-7H-pyrrolo[2,3-d]pyrimidin-2-amine; or
N-(7-(2-isopropoxyphenyl)-7H-pyrrolo[2,3-d]pyrimidin-2-yl)-3-methyl-2,3,4,5-tetrahydro-1H-benzo[d]azepin-7-amine.

20. A pharmaceutical composition comprising a compound of claim 1 or a pharmaceutically acceptable salt thereof, and a pharmaceutically acceptable carrier.

21. The composition of claim 20, further comprising an additional therapeutic agent selected from the group consisting of a chemotherapeutic or anti-proliferative agent, an anti-inflammatory agent, an immunomodulatory or immunosuppressive agent, an agent for treating a neurological disorder, an agent for treating cardiovascular disease, an agent for treating destructive bone disorders, an agent for treating liver disease, an anti-viral agent, an agent for treating blood disorders, an agent for treating diabetes, and an agent for treating immunodeficiency disorders.

22. A method of treating a disease, disorder, or condition mediated by AXL, comprising administering to a patient in need thereof a therapeutically effective amount of a compound of claim 1.

23. The method of claim 22, wherein the disease, disorder, or condition is cancer, asthma, chronic bronchitis, chronic obstructive pulmonary disease, adult respiratory distress syndrome, infant respiratory distress syndrome, cough, chronic obstructive pulmonary, adult respiratory distress syndrome, ulcerative colitis, Crohn's disease, hypersecretion of gastric acid, bacterial-, fungal-, or viral-induced sepsis or septic shock, endotoxic shock, spinal cord trauma, head injury, neurogenic inflammation, pain, reperfusion injury of the brain, psoriatic arthritis, rheumatoid arthritis, alkylosing spondylitis, osteoarthritis, inflammation, cytokine-mediated chronic tissue degeneration, thrombosis and the complications associated with thrombosis, macular degeneration, cataracts, diabetic retinopathy, glomerulonephritis, diabetic nephropathy, or renal plant rejection.

24. The method of claim 23, wherein the disease, disorder, or condition is a cancer.

25. The method of claim 24, wherein the cancer is lung cancer, myeloid leukemia, astrocytoma, uterine cancer, ovarian cancer, colorectal carcinoma, esophageal adenocarcinoma, glioblastoma, melanoma, prostate cancer, breast cancer, osteosarcoma, renal cell carcinoma, thyroid cancer, gastrointestinal stromal tumors, gastric cancer, hepatocellular carcinoma, kaposi sarcoma, pancreatic ductal adenocarcinoma, prostate cancer, or endometrial cancer.

26. (canceled)

27. (canceled)

28. (canceled)

29. (canceled)
